(19) United States
(12) Patent Application Publication
(10) Pub. No.: US 2023/0381511 A1
(43) Pub. Date: Nov. 30, 2023

(54) KINETIC CONTROL SUPPORT APPARATUS, KINETIC CONTROL SUPPORT METHOD, AND PROGRAM

(71) Applicant: NIPPON TELEGRAPH AND TELEPHONE CORPORATION, Tokyo (JP)

(72) Inventors: Arinobu NIIJIMA, Tokyo (JP); Yuki KUBO, Tokyo (JP)

(21) Appl. No.: 18/250,081

(22) PCT Filed: Nov. 6, 2020
(86) PCT No.: PCT/JP2020/041585

§ 371 Date: Apr. 21, 2023

(51) Int. Cl. A61N 1/36; G01L 1/10

(52) U.S. Cl. CPC A61N 1/36031; A61N 1/36003; G01L 1/10; A61N 1/0484

(57) ABSTRACT

A force control support device includes: a sound wave transmitting unit that causes a sound output device mounted on a user to transmit a first sound wave having a predetermined waveform; a sound wave receiving unit that causes a sound input device mounted on the user to receive a second sound wave based on the first sound wave; an estimation unit that estimates an amount of force being applied by the user based on the first sound wave and the second sound wave; and an electrical stimulation presentation unit that presents an electrical stimulation according to the amount of force estimated by the estimation unit from an electrode mounted on the user.

### TECHNICAL FIELD

The present disclosure relates to a force control support device, a force control support method, and a program.

### BACKGROUND ART

Conventionally, an exoskeleton glove as disclosed in Non Patent Literature 1 is known as an example of a device that supports control of the force of a fingertip, which is one of exercise skills of a human.

In addition, there is also known a method of controlling the force of a fingertip of a user by use of a technique of attaching an electrode to the skin surface and involuntarily contracting muscles by electrical stimulation (electrical muscle stimulation: EMS). For example, Non Patent Literature 2 discloses a force control support technique in which EMG data of a muscle of a forearm is measured by use of EMS and a myogenic potential sensor (EMG sensor), and an electrical stimulation is caused to flow through the muscle of the forearm according to the value of the EMG data, whereby each finger is bent and extended.

### CITATION LIST

### Non Patent Literature

Non Patent Literature 1: Takahashi, Nobuhiro, Shinichi Furuya, and Hideki Koike. “Soft Exoskeleton Glove with Human Anatomical Architecture: Production of Dexterous Finger Movements and Skillful Piano Performance.” IEEE Transactions on Haptics (2020).
Non Patent Literature 2: Nishida, Jun, and Kenji Suzuki. “BioSync: A paired wearable device for blending kinesthetic experience.” Proceedings of the 2017 CHI Conference on Human Factors in Computing Systems. 2017.
Non Patent Literature 3: Kubo, Yuki, et al. “AudioTouch: Minimally Invasive Sensing of Micro-Gestures via Active Bio-Acoustic Sensing.” Proceedings of the 21st International Conference on Human-Computer Interaction with Mobile Devices and Services. 2019.

### SUMMARY OF INVENTION

### Technical Problem

However, in the conventional techniques, there is a case where it is difficult to appropriately support force control by a user.

In one aspect, an object is to provide a technique capable of appropriately supporting force control by a user.

### Solution to Problem

In one proposal, a force control support device includes: a sound wave transmitting unit that causes a sound output device mounted on a user to transmit a first sound wave having a predetermined waveform; a sound wave receiving unit that causes a sound input device mounted on the user to receive a second sound wave based on the first sound wave; an estimation unit that estimates an amount of force being applied by the user based on the first sound wave and the second sound wave; and an electrical stimulation presentation unit that presents an electrical stimulation according to the amount of force estimated by the estimation unit from an electrode mounted on the user.

### Advantageous Effects of Invention

According to one aspect, it is possible to appropriately support force control by a user.

### BRIEF DESCRIPTION OF DRAWINGS

FIG. 1 is a diagram for describing a configuration of a force control support system according to an embodiment.

FIG. 2A is a diagram for describing an example of mounting positions of a microphone and a speaker according to the embodiment.

FIG. 2B is a diagram for describing an example of mounting positions of the microphone and the speaker according to the embodiment.

FIG. 2C is a diagram for describing an example of mounting positions of the microphone and the speaker according to the embodiment.

FIG. 3 is a diagram for describing a hardware configuration example of an information processing device according to the embodiment.

FIG. 4 is a diagram illustrating an example of a configuration of the information processing device according to an embodiment.

FIG. 5 is a flowchart for describing an example of processing of the information processing device according to the embodiment.

### DESCRIPTION OF EMBODIMENTS

Hereinafter, an embodiment of the present disclosure will be described with reference to the drawings.

<Overall Configuration>

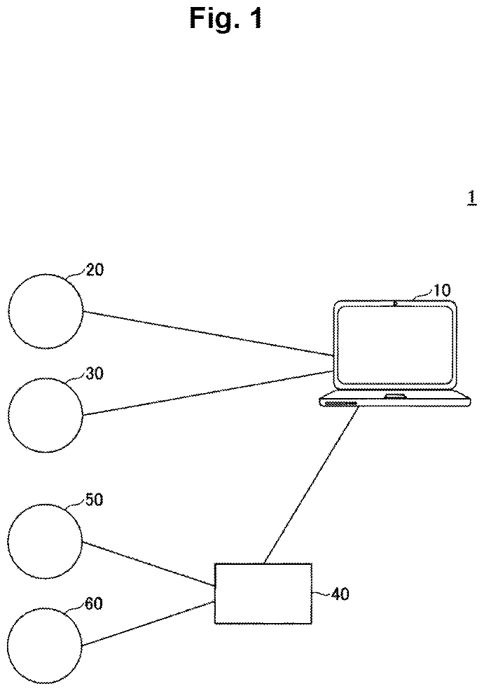

FIG. 1 is a diagram for describing a configuration of a force control support system 1 according to the embodiment. In the example of FIG. 1, the force control support system 1 includes an information processing device 10, a microphone 20 (an example of a “sound input device”), a speaker 30 (an example of a “sound output device”), a control device 40, an electrical stimulation device 50, and an electrical stimulation device 60. Note that the numbers of the electrical stimulation device 50, the electrical stimulation device 60, and the like are not limited to the example of FIG. 1.

The microphone 20 converts a collected sound into an audio signal and inputs the audio signal to the information processing device 10. The speaker 30 outputs a sound by an audio signal from the information processing device 10. The microphone 20 may include, for example, a piezoelectric element that converts a sound and an electric signal. Furthermore, the microphone 20 may be, for example, a capacitor microphone or the like.

The speaker 30 may include, for example, a piezoelectric element that converts an electric signal and a sound. Furthermore, the speaker 30 may be, for example, a vibration speaker or the like.

Hereinafter, an example of controlling the force of fingers of a user's hand will be described, but the technique of the present disclosure can be used not only for fingers of a hand but also for each part of a user such as fingers of a foot, an elbow, and a knee.

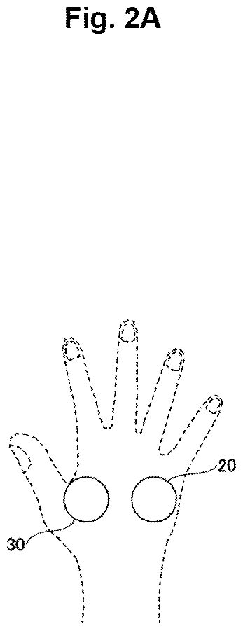

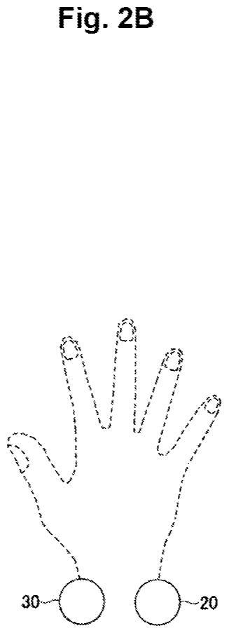

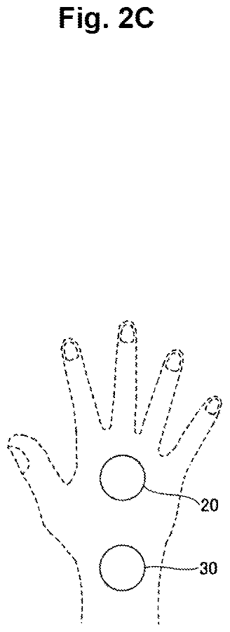

FIGS. 2A to 2C are diagrams each illustrating an example of mounting positions of the microphone 20 and the speaker 30 according to the embodiment. As illustrated in FIGS. 2A to 2C, for example, the microphone 20 and the speaker 30 may be mounted on (affixed to) a part of a hand other than fingers of a human (for example, the back of the hand, the wrist, or the like). In the example of FIG. 2A, the microphone 20 and the speaker 30 are mounted on the back of the hand. In the example of FIG. 2B, the microphone 20 and the speaker 30 are mounted on the wrist. In the example of FIG. 2C, the microphone 20 is mounted on the back of the hand, and the speaker 30 is mounted on the wrist.

The microphone 20 and the speaker 30 are only required to be attached so as not to be peeled off or lifted from the hand part. Note that, according to the present disclosure, sensing can be performed with high accuracy regardless of mounting positions, arrangements, orientations, and the like of the microphone 20 and the speaker 30.

The control device 40 controls the electrical stimulation device 50 and the electrical stimulation device 60 in accordance with an instruction from the information processing device 10. The control device 40 may be mounted on the human by, for example, a wristband or the like. The control device 40 may adjust the intensity of an electrical stimulation from the electrical stimulation device 50 and the electrical stimulation device 60 by pulse frequency modulation in which the frequency is changed, for example, between 0 and 200 Hz with a pulse width of 200 μs and a current of 10 mA.

The electrical stimulation device 50 and the electrical stimulation device 60 are electrodes or the like that give an electrical stimulation in accordance with an instruction from the control device 40. The electrical stimulation device 50 and the electrical stimulation device 60 may be mounted on (affixed to), for example, the forearm, the back of the hand, the palm, or the like, which is a part other than the fingers of the user.

The information processing device 10 uses the microphone 20 and the speaker 30 to perform active acoustic sensing, which uses a change in acoustic characteristics caused by a change in the orientation of the hand or the bulge of a muscle, thereby estimating the force of fingertips.

The information processing device 10 then controls the electrical stimulation device 50 and the electrical stimulation device 60 via the control device 40, and involuntarily contracts muscles related to bending or extending of the fingers by electrical muscle stimulation (EMS), thereby interactively supporting the control of the force of the fingertips. With this configuration, it is considered that, for example, the disclosed technique can be useful for supporting motor skill learning. For example, it is possible to support control of grip strength in gripping a racket or a ball.

The disclosed technique can also be used via a network. For example, an electrical stimulation is presented to each of users at remote locations while estimated values of their grip strengths are transmitted to and received from the users, so that it is possible to match their grip strengths.

Furthermore, the microphone 20 and the speaker 30 can be mounted on a first user, the electrical stimulation device 50 and the electrical stimulation device 60 can be mounted on a second user different from the first user, and an electrical stimulation can be given to the second user according to the grip strength of the first user. In this case, for example, the information processing device 10 may give the second user an electrical stimulation that causes the second user to apply a force equivalent to the grip strength of the first user to fingers of the second user. As a result, for example, it is possible to transmit, so to speak, a “grip strength” to a remote place.

<Hardware Configuration of Information Processing Device 10>

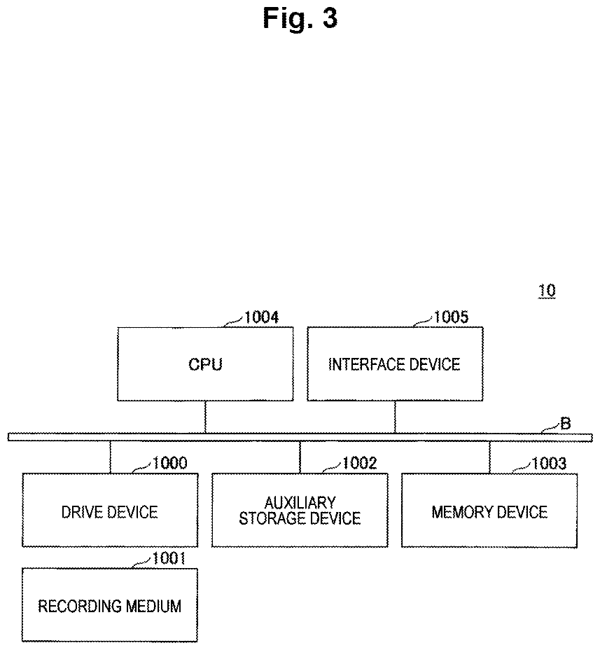

FIG. 3 is a diagram for describing a hardware configuration example of the information processing device 10 according to the embodiment. In the example of FIG. 3, the information processing device 10 includes a drive device 1000, an auxiliary storage device 1002, a memory device 1003, a CPU 1004, an interface device 1005, and the like, which are connected to each other via a bus B.

An information processing program for implementing processing in the information processing device 10 may be provided by a recording medium 1001. In this case, when the recording medium 1001 recording the information processing program is set in the drive device 1000, the information processing program is installed from the recording medium 1001 to the auxiliary storage device 1002 via the drive device 1000. However, the information processing program is not necessarily installed from the recording medium 1001, and may be downloaded from another computer via a network. The auxiliary storage device 1002 stores the installed information processing program and also stores necessary files, data, and the like.

In a case where an instruction to start a program is made, the memory device 1003 reads and stores the program from the auxiliary storage device 1002. The CPU 1004 executes processing in accordance with the program stored in the memory device 1003. The interface device 1005 is used as an interface for connecting to the network.

Note that an example of the recording medium 1001 includes a portable recording medium such as a CD-ROM, a DVD disk, or a USB memory. Furthermore, an example of the auxiliary storage device 1002 includes a hard disk drive (HDD), a flash memory, or the like. Each of the recording medium 1001 and the auxiliary storage device 1002 correspond to a computer-readable recording medium.

Note that the information processing device 10 may be implemented by, for example, an integrated circuit such as an application specific integrated circuit (ASIC) or a field-programmable gate array (FPGA).

<Configuration of Information Processing Device 10>

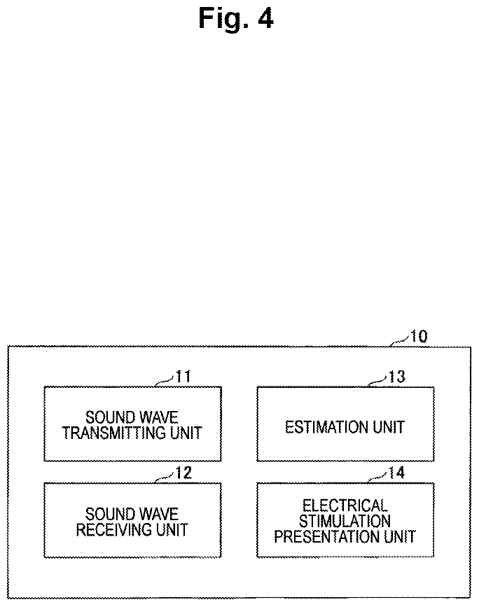

Next, a configuration of the information processing device 10 will be described with reference to FIG. 4. FIG. 4 is a diagram illustrating an example of the configuration of the information processing device 10 according to the embodiment.

The information processing device 10 includes a sound wave transmitting unit 11, a sound wave receiving unit 12, an estimation unit 13, and an electrical stimulation presentation unit 14. These units may be implemented by cooperation of one or more programs installed in the information processing device 10 and hardware such as the CPU 1004 of the information processing device 10.

The sound wave transmitting unit 11 causes the speaker 30 mounted on a user to transmit a first sound wave having a predetermined waveform. The sound wave receiving unit 12 causes the microphone 20 mounted on the user to receive a second sound wave based on the first sound wave.

The estimation unit 13 estimates the amount of force being applied by the user to a part such as fingers based on the first sound wave and the second sound wave.

The electrical stimulation presentation unit 14 supports control of the amount of force to be applied by the user to the part such as the user's own fingers by presenting an electrical stimulation according to the amount of force estimated by the estimation unit 13 from at least one electrode of the electrical stimulation device 50 and the electrical stimulation device 60 mounted on the user.

<Processing>

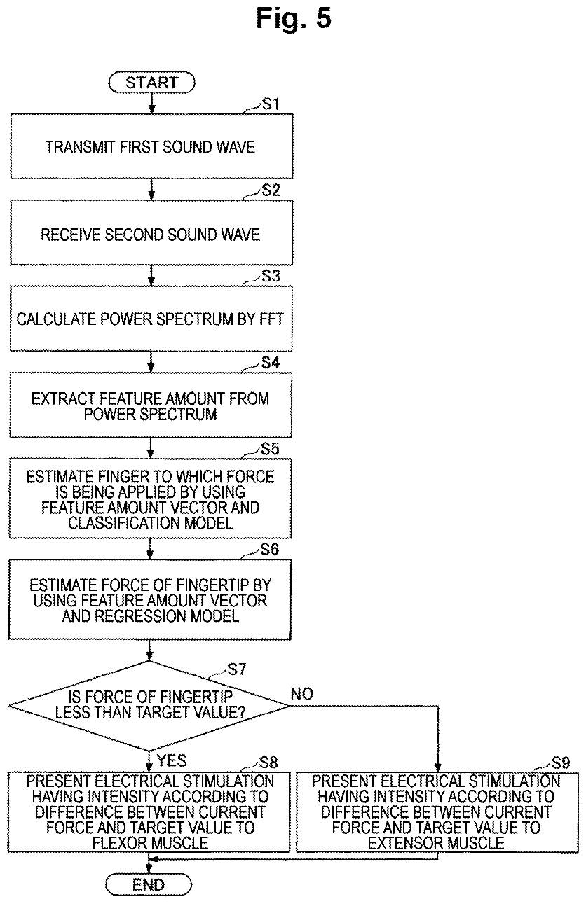

Next, an example of processing of the information processing device 10 according to the embodiment will be described with reference to FIG. 5. FIG. 5 is a flowchart for describing an example of the processing of the information processing device 10 according to the embodiment. The information processing device 10 may execute the following processing, for example, at a predetermined cycle.

In step S1, the sound wave transmitting unit 11 of the information processing device 10 causes the speaker 30 attached to the back of a hand of a human to transmit (output) a first sound wave. Here, for example, the information processing device 10 may cause the speaker 30 to output a sound waveform (sweep wave) by a sweep signal, which is a signal changed at a constant speed from a low frequency to a high frequency. In this case, for example, the information processing device 10 may output a waveform in which the frequency is linearly changed in a predetermined frequency range (for example, 20 kHZ to 40 kHZ) at a predetermined cycle (for example, 20 ms).

Subsequently, the sound wave receiving unit 12 of the information processing device 10 receives (acquires) a second sound wave collected by the microphone 20 attached to the back of the hand (step S2). As a result, the sound wave receiving unit 12 acquires an audio signal indicating a sound wave in which sound acoustic characteristics of a sound such as the sweep wave from the speaker 30 have been changed, which is caused by a change in the orientation of the hand or a change in the bulge of a muscle.

Subsequently, the estimation unit 13 of the information processing device 10 calculates a power spectrum of the audio signal collected by the microphone 20 (step S3). Here, for example, the information processing device 10 may perform fast fourier transform (FFT) for each predetermined number (for example, 4096) of samples to calculate the power spectrum.

Subsequently, the estimation unit 13 of the information processing device 10 extracts a feature amount from the calculated power spectrum (step S4). Here, for example, the information processing device 10 may extract a predetermined number (for example, 400) of peaks and calculate a feature amount vector of the extracted peaks.

Subsequently, the estimation unit 13 of the information processing device 10 estimates (infers) a finger to which force is being applied (an example of a part to be controlled) by using the feature amount vector and a classification model (step S5). Here, for example, the information processing device 10 may estimate the finger to which the user is applying force among a plurality of fingers of the user (identify which finger the user is applying force to) by using the classification model generated by a predetermined machine learning method with the feature amount vector as an input value. In this case, for example, the information processing device 10 may use a classification model by a machine learning method such as a support vector machine (SVM) and a neural network (NN) as the classification model. Note that the classification model is generated in advance and stored in the information processing device 10.

Subsequently, the estimation unit 13 of the information processing device 10 estimates the amount of force being applied by the user to the finger by using the feature amount vector and a regression model (step S6). Here, for example, the information processing device 10 may estimate the amount of force being applied by the user to the finger estimated in the processing of step S5 by using the regression model of machine learning generated by a predetermined machine learning method with the feature amount vector as an input value. In this case, for example, the information processing device 10 may use a regression model by a machine learning method such as support vector regression (SVR) and a neural network (NN) as the regression model. Note that the regression model is generated in advance and stored in the information processing device 10.

Subsequently, the electrical stimulation presentation unit 14 of the information processing device 10 determines whether or not the amount of force of the fingertip of the finger estimated in the processing of step S6 is less than a target value (threshold) (step S7). Here, for example, the information processing device 10 may determine the target value such that the transition of the amount of force being applied by the user to the fingertip or the like, which is sensed by the microphone 20 and the speaker 30, coincides with the transition of an amount of force set in advance. As a result, it is possible to perform interactive support according to the motion of the user.

In a case where the amount of force being applied by the user to the part to be controlled is less than the target value (YES in step S7), the electrical stimulation presentation unit 14 of the information processing device 10 gives (presents and applies) an electrical stimulation from the electrical stimulation device 50 or the electrical stimulation device 60 to a muscle that bends the finger in order to increase the force of the fingertip (step S8), and ends the processing.

Here, the electrical stimulation presentation unit 14 of the information processing device 10 selects the electrical stimulation device 50 or the electrical stimulation device 60 according to the part to be controlled from among the plurality of electrical stimulation devices 50 and 60 mounted on the user, and presents the electrical stimulation to the user from the selected electrical stimulation device 50 or electrical stimulation device 60. For example, in a case where the part to be controlled is the thumb, the information processing device 10 may select the electrical stimulation device 50 or the electrical stimulation device 60 mounted at a position corresponding to the flexor pollicis longus muscle, which is a muscle that bends the thumb, and give the electrical stimulation to the extensor pollicis longus muscle from the selected electrical stimulation device 50 or electrical stimulation device 60.

For example, the electrical stimulation presentation unit 14 of the information processing device 10 may give an electrical stimulation having an intensity according to a deviation degree (for example, a difference) of the force being applied by the user to the part to be controlled from the target value. In this case, for example, the information processing device 10 may increase the intensity of the electrical stimulation as a value obtained by subtracting the value of the force being applied by the user to the part to be controlled from the target value is larger. In this case, the information processing device 10 may perform, for example, proportional control, proportional-integral-differential controller (PID controller), or the like with the force being applied by the user to the part to be controlled as an input value.

On the other hand, in a case where the force being applied by the user to the part to be controlled is not less than the target value (NO in step S7), the electrical stimulation presentation unit 14 of the information processing device 10 gives an electrical stimulation from the electrical stimulation device 50 or the electrical stimulation device 60 to a muscle that extends the finger in order to reduce the force of the fingertip (step S9), and ends the processing.

Here, the electrical stimulation presentation unit 14 of the information processing device 10 selects the electrical stimulation device 50 or the electrical stimulation device 60 according to the part to be controlled from among the plurality of electrical stimulation devices 50 and 60 mounted on the user, and presents the electrical stimulation to the user from the selected electrical stimulation device 50 or electrical stimulation device 60. For example, in a case where the part to be controlled is the thumb, the information processing device 10 may select the electrical stimulation device 50 or the electrical stimulation device 60 mounted at a position corresponding to the extensor pollicis longus muscle, which is a muscle for extending the thumb, and give the electrical stimulation to the extensor pollicis longus muscle from the selected electrical stimulation device 50 or electrical stimulation device 60.

Here, for example, the information processing device 10 may give an electrical stimulation having an intensity according to a deviation degree (for example, a difference) of the target value from the force being applied by the user to the part to be controlled. In this case, for example, the information processing device 10 may increase the intensity of the electrical stimulation as a value obtained by subtracting the target value from the value of the force being applied by the user is larger. In this case, the information processing device 10 may perform, for example, proportional control, proportional-integral-differential controller (PID controller), or the like with the force being applied by the user as an input value.

<Modification 1>

In the example described above, an example has been described in which the finger to which the user is applying force is determined as a part to be controlled in the processing of step S5 in FIG. 5. Alternatively, the processing of steps S1 to S9 in FIG. 5 may be performed on a plurality of predetermined fingers (for example, all five fingers) of the user. As a result, for example, it is possible to control the force by stimulating a muscle of each finger of the user while a video for sports training is reproduced and the user is allowed to perform the same motion as a model of the video.

<Modification 2>

At least a part of the functional units of the information processing device 10 may be implemented by, for example, cloud computing provided by one or more computers. In this case, for example, the estimation unit 13 and the electrical stimulation presentation unit 14 may be provided in an external information processing device.

Furthermore, the information processing device 10 and the control device 40 may be configured as an integrated device. Furthermore, at least a part of the functional units of the information processing device 10 may be provided in the control device 40.

### Effects of Present Disclosure

For example, in a case where sensing or the like is performed by use of an exoskeleton glove or the like, a movable range of a finger or the like is restricted, or a tactile sense of a fingertip is hindered. In addition, when the state (force) of bending or extension of a finger is observed (sensed) by an electric signal in a case where bending or extension of each finger is urged by use of the EMS technology, the electric signal of EMS becomes a cause of a noise for sensing of the state (force) of bending or extension of the finger. As a result, the accuracy of sensing is reduced.

According to the present disclosure, it is possible to appropriately support force control by a user. Furthermore, according to the present disclosure, it is possible to appropriately support control of the force of a fingertip, for example, without a glove or the like mounted on the fingertip. In addition, according to the present disclosure, it is possible to appropriately measure the state (force) of bending or extending of a finger even while bending or extending of each finger is urged by use of the EMS technology.

Therefore, for example, in a sport played with a tool such as a tennis racket, a golf club, or a ball, the disclosed technique can also be used to teach a user the ideal grip strength. In addition, for example, in the fields of medical care (medical treatment) and nursing for humans and animals, the disclosed technique can also be used for teaching a beginner a subtle difference in force adjustment when a doctor performs palpation to know a medical condition by touching each part of a body of a patient (human or animal) with fingers.

Although the embodiment of the present invention has been described in detail above, the present invention is not limited to such a specific embodiment, and various modifications and changes can be made within the scope of the gist of the present invention described in the claims.

### REFERENCE SIGNS LIST

1 Force control support system
10 Information processing device
11 Sound wave transmitting unit
12 Sound wave receiving unit
13 Estimation unit
14 Electrical stimulation presentation unit
20 Microphone
30 Speaker
40 Control device
50 Electrical stimulation device
60 Electrical stimulation device

1. A force control support device comprising:
a memory; and
a processor coupled to the memory and configured to
cause a sound output device mounted on a user to transmit a first sound wave having a predetermined waveform;
cause a sound input device mounted on the user to receive a second sound wave based on the first sound wave;
estimate an amount of force being applied by the user based on the first sound wave and the second sound wave; and
present an electrical stimulation according to the estimated amount of force from an electrode mounted on the user.

2. The force control support device according to claim 1, wherein,
the processor is configured to select an electrode mounted on a predetermined part of the user from among a plurality of electrodes, and present an electrical stimulation that supports control of an amount of force to be applied by the user from the selected electrode.

3. The force control support device according to claim 1, wherein
the processor is configured to present an electrical stimulation that supports control of an amount of force to be applied by the user to a finger via an electrode mounted on a part other than the finger of the user.

4. The force control support device according to claim 1, wherein
the processor is configured to estimate a finger to which the user is applying force among a plurality of fingers of the user by a classification model, and estimate an amount of force being applied by the user to the finger by a regression model.

5. A force control support method performed by a force control support device, the force control support method comprising:
causing a sound output device mounted on a user to transmit a first sound wave having a predetermined waveform;
causing a sound input device mounted on the user to receive a second sound wave based on the first sound wave;
estimating an amount of force being applied by the user based on the first sound wave and the second sound wave; and
presenting an electrical stimulation according to the estimated amount of force from an electrode mounted on the user.

6. A non-transitory computer-readable recording medium storing a program for causing a computer to execute processing comprising:
causing a sound output device mounted on a user to transmit a first sound wave having a predetermined waveform;
causing a sound input device mounted on the user to receive a second sound wave based on the first sound wave;
estimating an amount of force being applied by the user based on the first sound wave and the second sound wave; and
presenting an electrical stimulation according to the estimated amount of force from an electrode mounted on the user.
